(19) United States
(12) Patent Application Publication
(10) Pub. No.: US 2014/0276912 A1
(43) Pub. Date: Sep. 18, 2014

(54) SURGICAL DEVICE HAVING AN INNER THREAD AND A SHARPENING DEVICE FOR SUCH A DEVICE

(71) Applicant: Andre H. SAAD, Roslyn, NY (US)

(72) Inventor: Andre H. SAAD, Roslyn, NY (US)

(21) Appl. No.: 13/839,252

(22) Filed: Mar. 15, 2013

(51) Int. Cl. A61B 17/3205

(52) U.S. Cl. CPC A61B 17/32053
USPC 606/114

(57) ABSTRACT

There is disclosed a medical instrument for removing tissue from a body comprising a body, and a shaft coupled to the body, wherein the shaft is substantially cylindrical. This shaft has an inner surface and an outer surface and a sharpened end. This shaft has a radius, a diameter and a longitudinal axis. The inner surface of the shaft has a thread extending along a substantial portion of the shaft and a hollow inner region on the shaft. There is also a process for removing tissue which can include using this morcellator with the thread on the inside of the shaft. There can also be a morcellator sharpener which includes a conical body formed as a substantially hollow cone having both an inner grinding surface and an outer grinding surface wherein the sharpener is rotatably mounted to the stand.

### BACKGROUND OF THE INVENTION

At least one embodiment of the invention relates to a surgical device that can have a thread, disposed inside of a cylindrical shaft. A driver or motor can be used to drive the shaft in a rotatable manner to extract tissue samples or tissue from a person. This type of device can be in the form of a modified morcellator. There can also be a sharpener for the morcellator and a process for using the modified morcellator or the sharpener as well.

### SUMMARY OF THE INVENTION

At least one embodiment of the invention relates to a medical instrument comprising a body, and a shaft coupled to the body wherein the shaft is substantially cylindrical. In at least one embodiment, this medical instrument can be a morcellator. This shaft has an inner surface and an outer surface and a sharpened end. In at least one embodiment, the shaft has a radius, a diameter and a longitudinal axis. An inner surface of the shaft has a thread extending along a substantial portion of the shaft and a hollow inner region in the shaft. The sharpened end can be sharpened on the inside, the outside or on both the inside and the outside of the end.

The body has a base and a handle. There can be a motor disposed in the body and configured to turn said shaft in a rotatable manner. This turning of the shaft can be in an oscillating manner. In at least one embodiment, the handle extends substantially perpendicular to the base. In at least one embodiment, the shaft is made of metal and said body is made from a non-metal material. The thread can extend along a substantial portion of the shaft and can extend along at least 30% of the length of the shaft in one embodiment, at least 50% of the shaft in another embodiment, at least 80% of the length of the shaft, or along the entire length of the shaft.

The threads can be of different sizes within the shaft.

There can also be a process for removing tissue comprising the steps of using a surgical device such as a morcellator having a shaft, with an inside surface and an outside surface, wherein the inside surface has a thread extending along a substantial length of the shaft. This shaft can have a thread that is in a helical pattern as well. The process also includes the step of rotating the shaft to cut a piece of tissue inside of a body. Next, the process includes extracting a piece of tissue using the rotation of the shaft, with the inside threading gripping the piece of tissue to draw the tissue up into the shaft and away from a user's body. There can also be the step of extending forceps through the shaft to draw tissue samples through the shaft. This process can also include the step of drawing the piece of tissue up through the shaft via the threading.

There can also be a morcellator sharpener comprising a body formed as a substantially hollow cone having an outer grinding surface and an inner grinding surface, a stand, and a support base. The stand can be configured as substantially cylindrical in shape and the stand is configured to fit at least partially inside of the support base. The outer grinding surface has a roughened surface configured to sharpen an inside surface of an end of a morcellator shaft. The inner grinding surface has a roughened surface configured to sharpen an outside surface of the end of the morcellator shaft.

One of the benefits of the invention relating to the threading on the inside surface of the shaft, is that this provides an inner channel for drawing the tissue up and out of the body. The circular cutting motion of the end of the shaft of the blade draws the tissue up in a spiral motion. The threading thereby provides an extra surface for the tissue to grab and be drawn up through the shaft. The threading thereby reduces tissue drag and tissue pop-offs. In addition, it reduces surgical time as well and surgeon fatigue as the tissue is more consistently and expeditiously drawn up during surgery.

Similarly, the addition of the sharpener allows for the blade or free end of the shaft to be kept consistently sharp during surgery. For example, during surgery, the blade can dull when used on a person's body. Therefore, the blade should be sharpened even during surgery so that the end point of the shaft or the blade can be kept sharp and used to cut into a tissue inside of a person's body. If during surgery, the blade which is positioned on the tip of the shaft becomes dull, then the shaft would have to be replaced. This replacement of the shaft during surgery can take up valuable time during surgery. Alternatively, the entire morcellator would have to be discarded and a new morcellator used. Therefore, with the addition of this sharpener, the blade can be sharpened during surgery and then put back in use to remove more tissue from a person's body.

### BRIEF DESCRIPTION OF THE DRAWINGS

Other objects and features of the present invention will become apparent from the following detailed description considered in connection with the accompanying drawings which disclose at least one embodiment of the present invention. It should be understood, however, that the drawings are designed for the purpose of illustration only and not as a definition of the limits of the invention.

In the drawings, wherein similar reference characters denote similar elements throughout the several views:

FIG. 1 is a side perspective view with a cut-away showing a thread extending along an inside surface of the shaft;

FIG. 2 is a side perspective view showing a cut-away of the shaft showing a slightly larger thread along the inside surface of the shaft;

FIG. 3A is a side perspective view of another embodiment;

FIG. 3B is a side perspective view of another embodiment;

FIG. 4 is a first side perspective view of the sharpener;

FIG. 5 is a second side perspective view of the sharpener;

FIG. 6A is a flow chart of the process of using any one of the devices shown in FIGS. 1-3 and optionally the sharpener in FIGS. 4 and 5;

FIG. 6B is a side cross-sectional view of one portion of a shaft of the device;

FIG. 6C is a side cross-sectional view of another embodiment of the shaft;

FIG. 6D is a side cross-sectional view of another embodiment of the shaft;

FIG. 7 is a perspective view of one embodiment;

FIG. 8 is a bottom perspective view of the embodiment of FIG. 7;

FIG. 9 is a side view of the embodiment of FIG. 7;

FIG. 10 is a side perspective view of another embodiment;

FIG. 11 is a side perspective view of the embodiment of FIG. 10 with the sharpener positioned in an opposite orientation;

FIG. 12A is a bottom-side perspective view of another embodiment; and

FIG. 12B is a top-side perspective view of the embodiment shown in FIG. 12A.

### DETAILED DESCRIPTION OF PREFERRED EMBODIMENTS

Turning in detail to the drawings, FIG. 1 discloses a perspective view of a first embodiment of the device 10. In this view, there is shown a body 12 having a first section extending along a first axis 11.1 and a second section extending along an axis 11.2. Axis 11.2 is substantially perpendicular to axis 11.1. Coupled to body 12 is a handle 14. In addition, coupled to body 12 is a trigger 16. Trigger 16 is for selectively operating a motor 19 disposed in body 12. Motor 19 is configured to selectively drive shaft 20 which is coupled to body 12. Shaft 20 can be driven in a rotatable manner, wherein shaft 20 can be rotated around axis 21, or oscillated around axis 21. Shaft 20 is a substantially cylindrical shaft having a hollow center and a body section 22. There is a thread 24 which extends along axis 21 inside of shaft 20. Thus, shaft 20 includes the inside surface 22.1, and an outside surface 22.2. Inside surface 22.1 is for receiving thread 24, while outside surface 22.2 can be substantially smooth. At an end of shaft 20 is a sharpened end 26.

As shown, shaft 20 includes a length which extends along longitudinal axis 21 and a diameter 22d which extends across the mouth of the opening of the shaft. Sharpened end 26 can be sharpened from the outside to create an outside sharpened surface 28 or have an inside sharpened surface 29 as well. A section 26.1 which is shown in greater detail in FIGS. 6B-6D shows the different ways in which end can be sharpened either with a double bevel, or a single bevel, either on the outside face 28 or on the inside face 29.

In this embodiment, the threads 24 can form a continuous spiraling line, which are threaded throughout the entire length of the shaft, or just a portion of the length of the interior or inside surface 22.1 of shaft 20. The extent of this threading can be 20% or greater of the length, 40% or greater of the length, greater than 50% of the length, greater than 90% of the length or the entire length. The size of the threads are such that they are sufficient to grip and hold tissue being extracted. The threads 24 can be configured such that they convey material drawn up through the shaft 20 when the shaft 20 is rotating. For example, if a sample such as a tissue sample is drawn up into an interior region of shaft 20, the rotation of shaft 20 along with threads 24 grips and holds the material or tissue sample and draws in this tissue sample up through shaft 20 and away from a person's body. Thus, thread 24 acts as a conveyor belt for these tissues as they are drawn up and through the shaft 20.

This shaft can continue with an opening uninterrupted all the way to the back of the device such that it has an open back end 29a. This open back end and continuous shaft allows for the introduction of forceps (not shown) or other medical instruments into the shaft so that a user such as a surgeon can draw flesh through the shaft, up into the shaft and let the threads of the shaft take hold of the tissue samples.

In addition, because this shaft is hollow, additional instruments such as forceps can be inserted through the shaft. These forceps can be inserted through the shaft after the sharpened end 26 creates an incision in a patient's tissue.

As shown in FIG. 3A, there is a cross-sectional side view of the shaft 20a including shaft body 22a. Extending inward from shaft body 22a are threads 24a. FIG. 3A shows a first embodiment, wherein the threads are approximately the size of ⅛ of the diameter of the opening of the shaft. Therefore, with this embodiment, each thread extends in ⅛ of the extent of the diameter such that two opposing threads would occupy approximately ¼ of the opening of the shaft. At one end of the shaft is the opening for receiving tissue. At the opposite end of the shaft is an open end 29a allowing user such as a surgeon to insert medical instruments such as forceps therein. The introduction of forceps allows the surgeon to extend the forceps through the shaft 20 or 20a, or 20b out the opposite side of the shaft, and into user's body to draw tissue into the shaft. As the shaft is rotating, these threads such as threads 24a which extend into the hollow region of the shaft, can be used to grab the tissue and draw it up into the shaft.

FIG. 3B is a side cross-sectional view of a second embodiment of a shaft 22b. In this embodiment, the individual thread 24b extends inward proximally 1/16 of the diameter of the opening of the shaft. Thus, the thread 24b, along with an opposite thread 24b, extend a combined amount of ⅛ of the open diameter into the opening of the shaft. Either one of shafts 20a or 20b can be used in place of shaft 20 shown in FIG. 1.

While two embodiments showing varying sizes of threads have been shown, any other suitable sizes can also be used. However, the extent of the threads must be limited to allow a user such as a surgeon to use medical instruments therein (in the center of the shaft) during surgery. These threads are formed in a spiral, helical fashion such that the rotation of shaft 20 or shaft 20a, or 20b results in the threads such as threads 24, 24a, or 24b rotating as well which creates an angled path to draw material such as tissue up into the shaft.

FIG. 4 is a perspective view of the sharpener for the medical instrument or device 10 or 30. As disclosed above this medical device could be a morcellator. Thus, as shown, there is shaft 20 with inside sharpened surface 29. Sharpener 50 includes an outside sharpening surface 52 which has a substantially conical shape. Sharpening surface 52 starts in the first end 52a which has a substantial point and extends out such that it is radially wider at its base section 52b. At this base section 52b, there is a bottom surface 56. In addition, inside of sharpener 50 is an inside sharpening surface 54. The inside of the sharpener 50 is hollow as shown by hollow region 58. Thus, the sharpener 50 can be used to sharpen both the inside surface 29 of shaft 20 as well as the outside surface 28 of shaft 20.

For example, as shown in FIG. 5, sharpener 50 has an inside surface 54 which is also substantially conical shape. However, the sharpening of this morcellator which includes shaft 20 is designed to sharpen outer sharpened surface 28 rather than inner sharpened surface 29. Thus, when a user inserts shaft 20 inside of the hollow region 58 of sharpener 50, outside sharpened region 28 contacts inner sharpened surface 54 to sharpen the device.

To sharpen this morcellator type device, the user could initially sharpen the inside sharpened surface 29 by putting the shaft 20 onto the sharpening device/sharpener 50. Then, the user would activate the device by rotating shaft 20 around sharpening device 50 such that inside sharpened surface 29 contacts and wears against outside sharpening surface 52. As shaft 20 is rotating, inside sharpened surface 29 becomes sharpened by sharpening surface 52 wearing against inside sharpened surface 29.

Additionally, to sharpen the device on the outside, the user would insert shaft 20 into hollow region 58 and activate the morcellator by rotating shaft 20 such that outside sharpened surface 28 wears against inner sharpening surface 54 thereby sharpening the end of this device 26 to a point (See FIG. 6B). Thus, during operation, a surgeon who is confronted with a dull edge of medical instruments, such as a morcellator can during the operation, sharpen the end of this morcellator without losing much valuable time during surgery. The use of the sharpener 50 also would extend the life of these medical instruments, particularly the shaft 20.

Alternatively, a surgeon could sharpen these ends of the medical instruments after surgery and then treat these shafts in a sonic bath or heated bath to sterilize these medical instruments as well.

FIG. 6A is a flowchart of the process for using any one of the embodiments of FIG. 1, 2, or 3, and also includes using the sharpeners of FIGS. 4 and 5.

For example, the process starts in step S1 which includes providing a medical instrument having a hollow shaft with an inside surface having a thread. An example of this type of medical device is shown in FIG. 1 as embodiment 10.

Next step S2 includes process of rotating shaft 20, to cut a tissue within the body. As discussed above, motor 19 can be activated when trigger 16 is pressed to drive motor 19 to rotate shaft 20.

Next, an optional step S3 includes using forceps to extract tissue through hollow shaft 20, by drawing tissue cut by the shaft or blade, away from the user's body up through any one of these shafts.

Step 4 includes extracting a piece of tissue via rotation of the shaft with the inside threading 24 gripping the piece of tissue to draw the tissue up the shaft. Because the rotational shaft drives either an inner thread, to rotate, the spiral configuration of either the thread 24 under rotation, results in the drawing up of tissue through the shaft. Thus, the use of the threading 24, results in a surface or geometry which is configured to draw tissue samples or body tissue up through the shaft during operation. Thus, with these designs the threading thereby reduces tissue drag or tissue popoffs. In addition, the use of these threads reduces the time for operation as well as the surgeon fatigue during operation or surgery.

In a next step S5 the end of the shaft 20, forming a blade is rotated against the sharpener 50, either against the outside surface 52 or the inside surface 54 to sharpen either the inside surface of the blade 29, or the outside surface of the blade 28 respectively.

FIG. 6B is a side cross-sectional view of one portion of a shaft 20 taken at section 26.1 (See FIG. 1) of the device. This view shows a cross-sectional view of one small portion of a shaft 20 which includes a sharpened end 26. Sharpened end 26 is formed by an outside sharpened surface 28 and an inside sharpened surface as described above. In addition, in this cross-sectional view there are also shown threads 24. Thus, in this view the end of the shaft has a double beveled edge which is beveled on both sides of the device.

FIG. 6C is a side cross-sectional view of another

FIG. 7 is a perspective view of another embodiment, wherein it shows a morcellator shaft 20 of a morcellator 10 which is configured to be sharpened by sharpener 50. Sharpener 50 has a first end 52a second end 52b which has a larger diameter. The shape of sharpener 50 is that of a cone or substantially that of a cone. The conical sharpener as shown in FIG. 4 and in FIG. 5 has a hollow interior 54 which is configured to receive shaft 20 as well. In addition, as shown there is a stand 70 which is a substantially cylindrical shaped stand, and is coupled to sharpener 50. Stand 70 forms an annular ring which is coupled to sharpener 50. This stand 70 has a first side 70a (see FIG. 9), and a second side 70b shown in the bottom perspective view, and an outer rim or ring 70c. Essentially, the stand 70 forms a doughnut shaped stand. As shown in this view, shaft 20 can be rotated about its longitudinal axis which is shown by arrow 20b.

As shown in FIG. 8, there is a bottom perspective view of the embodiment shown in FIG. 7. In this view, there is shown sharpener 50 having pointed end 52a, stand 70, having outside ring 70c as well as bottom surface or face 70b. End section 52b is shown as coupled to stand 70.

FIG. 9 shows a side view of sharpener 50 coupled to stand 70, which shows outer ring 70c as well as a top surface or face 70a.

Thus this type stand allows for stabilization of sharpener 50 so the sharpener 50 can rest on a tabletop or support surface and be used to sharpen a morcellator blade. This stand 70, can also serve as a handle wherein a user such as a surgeon or a surgical assistant can hold the sharpener stand and sharpener 50 in one hand while a surgeon or surgical assistant sharpens the morcellator blade. The surfaces 70a, 70b, and 70c create an easy surface or platform for a user to grip the stand 70 and hold the sharpener 50 in place.

FIG. 10 shows side perspective view of sharpener 50 coupled to stand 70 with first surface 70a being shown as well as outside rim or ring 70c also being shown. There is also shown a plurality of support tabs 83a, 83b, and 83c coupled to a support base 80 having a top rim 82. These support tabs 83a, 83b, and 83c are recessed at a position below top rim 82. This allows for stand 70 to be placed inside of support base 80 while being supported by an outer rim 84 of support base 80. Thus, in the first position shown in FIG. 10, the user can place sharpener 50 with stand 70 inside of support base 80 and support this sharpener as well stand 70. Thus, a user sharpening the inside of shaft 20 of morcellator 10 can then use both hands to sharpen this shaft. Alternatively, as shown in FIG. 11 a user can reorient sharpener 50 and stand 70 such that pointed end 52 a extends into a hollow region of support base 80. Surface 70a is now supported by support tabs 83a, 83b, and 83c with stand 70 resting inside of outer rim 84 and supported therein. In this position, the user can insert shaft 20 of device 10 into the inside hollow region 54 of sharpener 50 as shown in FIG. 5. In this way, support base 80 holds sharpener 50 in place so that a user such as a surgeon can sharpen end 26, and in particular, outer sharpened surface 28.

FIG. 12A is a bottom-side perspective view of another embodiment. In this embodiment, there shown a different style sharpener 90, which is a frusto-conical sharpener having an open top and 91. Sharpener 90 is coupled to stand 70 and has an end 94 forming a hollow cylindrical guide for guiding a morcellator shaft such as shaft 20 (See FIG. 1) therein. Guide 94 has threading 95 as well as a hollow interior 96. Hollow interior 96 is configured to receive a morcellator shaft such as shaft 20 when it's inserted therein. In addition, threading 95 allows for adjustability of sharpener 90 relative to stand 70. Thus, as shown, end 94 is shown screwed in FIG. 12A such that stand 70 is positioned at least partially along longitudinal axis 97 of sharpener 90. In this way, the threaded region is positioned below or substantially on second side 70b of stand 70.

As shown in FIG. 12B, there is a side perspective view of the embodiment shown in FIG. 12A. In this view, stand 70 is shown position at a bottom region of and 94 with threaded region 95 being positioned on an opposite side of surface 70a. in this view, threading region 95 is positioned such that a substantial portion of this threaded region is positioned on the side of top surface 70a of stand 70.

Thus, with the design of the stand 70 as well as the support base 80, there is a stable sharpening system which can be used to sharpen a surgical instrument such as a morcellator 10 which can be easily used to sharpen both an inside surface 29 of a hollow shaft 20, as well as an outside surface 28 of shaft 20 with a single sharpening device.

With both a combination of the threaded shaft, and the sharpening system including sharpener 50, stand 70, and support base 80, a surgeon can perform a surgical procedure using a single morcellator shaft in an efficient manner, resulting in less surgeon fatigue, shorter time for surgery, and potentially reduced chance of any surgical error.

Similarly, by providing a sharpener for the blade of a shaft, this allows a surgeon to keep a blade sharp continuously during surgery which will also reduce surgeon fatigue and reduce the amount of time necessary for a surgery.

Accordingly, while at least one embodiment of the present invention has been shown and described, it is to be understood that many changes and modifications may be made thereunto without departing from the spirit and scope of the invention as defined in the appended claims.

What is claimed is:

1. A medical instrument comprising:
a) a body;
b) a shaft coupled to said body, said shaft being substantially cylindrical, wherein said shaft has an inner surface and an outer surface and a sharpened end, wherein said shaft has a radius, a diameter and a longitudinal axis, and wherein said inner surface of said shaft has a thread extending along a substantial portion of said shaft and a hollow inner region on said shaft.

2. The medical instrument as in claim 1, wherein said body has a base and a handle.

3. The medical instrument as in claim 1, further comprising a motor disposed in said body and configured to turn said shaft in a rotatable manner.

4. The medical instrument as in claim 1, wherein said motor is configured to drive said shaft in an oscillating manner.

5. The medical instrument as in claim 2, wherein said handle extends substantially perpendicular to said base.

6. The medical instrument as in claim 1, wherein said sharpened end is sharpened on said outside surface of said shaft.

7. The medical instrument as in claim 1, wherein said sharpened end is sharpened on said inside surface of said shaft.

8. The medical instrument as in claim 1, wherein said sharpened end is sharpened on both said outside surface and said inside surface of said shaft.

9. The medical instrument as in claim 1, wherein said shaft is made of metal and said body is made from a non-metal material.

10. The medical instrument as in claim 1, wherein said thread extending along a substantial portion of said shaft extends along at least 30% of the length of the shaft.

11. The medical instrument as in claim 1, wherein said thread extending along a substantial portion of said shaft extends along at least 50% of the length of the shaft.

12. The medical instrument as in claim 1, wherein said thread extending along a substantial portion of said shaft extends along at least 80% of the shaft.

13. The medical instrument as in claim 1, wherein the medical instrument is a morcellator.

14. The medical instrument as in claim 1, wherein said thread has a threadpath within said inside surface of said shaft, wherein the threadpath extends in a helical manner along said shaft.

15. The medical instrument as in claim 1, wherein
said body has a base and a handle and said handle extends substantially perpendicular to said base, wherein said shaft is made from a metallic material and said body is made from a non-metallic material;
and wherein the instrument further comprises a motor disposed in said body and configured to turn said shaft in a rotatable manner;
wherein said shaft has a sharpened end that is sharpened on both said outside surface and said inside surface of said shaft; and
wherein said thread extending along a substantial portion of said shaft extends along at least 80% of the shaft; and
and wherein the medical instrument is a morcellator.

16. A process for removing tissue comprising:
a) using a morcellator having a shaft with an inside surface and an outside surface wherein said inside surface has a thread extending along a substantial length of said shaft;
b) rotating said shaft to drive said shaft to cut a piece of tissue within the body; and
c) extracting a piece of tissue using the rotation of the shaft, and the inside threading gripping the piece of tissue to draw the tissue up into the shaft.

17. The process as in claim 16, further comprising extending forceps through the shaft to draw samples through the shaft.

18. The process as in claim 17, further comprising the step of drawing the piece of tissue up through the shaft via the threading.

19. A morcellator sharpener comprising:
a) a body formed as a substantially hollow cone having an outer grinding surface and an inner grinding surface;
b) a stand;
c) a support base having at least one support, wherein said at least one support is positioned in a recessed region of one end of said support base such that said stand is configured to sit inside of said support base and be supported by said support base, wherein said stand is configured to be positioned in at least a first orientation within said support base and in at least a second orientation in said support base to allow a user to sharpen an inside surface of a shaft of a morcellator and an outside surface of a shaft of the morcellator.

20. The sharpener as in claim 19, wherein the sharpener is frusto-conical in shape and has a roughened surface on both an inside surface and an outside surface of the sharpener, and wherein the sharpener has an end that is substantially cylindrical comprising at least one thread on an exterior surface, wherein said sharpener is configured to screw into said stand, and wherein said at least one support base comprises a substantially cylindrical base.
